(19) United States
(12) Patent Application Publication
(10) Pub. No.: US 2009/0069763 A1
(43) Pub. Date: Mar. 12, 2009

(54) Flat Expandable Effusion Drain

(76) Inventors: Paul DiCarlo, Middleboro, MA (US); Jon T. McIntyre, Newton, MA (US)

Correspondence Address: FAY KAPLUN & MARCIN, LLP, 150 BROADWAY, SUITE 702, NEW YORK, NY, 10038, US

(21) Appl. No.: 12/207,988

(22) Filed: Sep. 10, 2008

Related U.S. Application Data
(60) Provisional application No. 60/971,444, filed on Sep. 11, 2007.

(51) Int. Cl. A61F 5/44

(52) U.S. Cl. 604/328

(57) ABSTRACT

An effusion drain includes a fluid collection element moving between an operative shape for receiving a fluid and a reduced volume shape for connecting to a fluid drain and a compressible member biasing the fluid collection element to the operative shape, the compressible member being incorporated into the fluid collection element such that release of a compression on the compressible member expands the fluid collection element to the operative shape.

### PRIORITY CLAIM

This application claims priority to U.S. Provisional Application Ser. No. 60/971,444, entitled “Flat Expandable Effusion Drain,” filed Sep. 11, 2007. The Specification of the above-identified application is incorporated herewith by reference.

### BACKGROUND OF THE INVENTION

Fluids often build up at or near trauma sites. If not removed, the fluid may cause swelling, pain and/or other complications. To minimize these complications, drains are often installed near the trauma sites (e.g., surgical openings, injuries, etc.) to provide an exit path for the fluid.

Effusion drains have a distal end that is inserted into the body near the trauma site with a proximal end open outside the body. These drains often include a drainage bulb at the proximal end to collect fluids and to create an initial suction assisting in removing fluids through the drain. After implantation, these effusion drains are generally maintained by the patient.

### SUMMARY OF THE INVENTION

In one aspect, the present invention is related to an effusion drain comprising a fluid collection element moving between an operative shape for receiving a fluid and a reduced volume shape for connecting to a fluid drain and a compressible member biasing the fluid collection element to the operative shape, the compressible member being incorporated into the fluid collection element such that release of a compression on the compressible member expands the fluid collection element to the operative shape.

### BRIEF DESCRIPTION OF THE DRAWINGS

FIG. 1 is a schematic drawing showing a conventional effusion drain with a drainage bulb;

FIG. 2 is a diagram of an effusion drain according to the present invention in an open configuration;

FIG. 3 is a diagram of the effusion drain shown in FIG. 2 in a closed configuration;

FIG. 4 is a diagram showing a second embodiment of the effusion drain according to the invention;

FIG. 5 is a diagram of an inflatable drainage bag according to an embodiment of the invention,

FIG. 6 is a diagram showing a chest cavity with a pleural effusion treated using the embodiment shown in FIG. 5;

FIG. 7 is a diagram of a drainage bag according to another embodiment of the invention,

FIG. 8 is a diagram of a drainage bag according to an alternate embodiment of the invention; and

FIG. 9 is a diagram of a drainage bag according to a further embodiment of the invention.

### DETAILED DESCRIPTION

The present invention may be further understood with reference to the following description and the appended drawings, wherein like elements are referred to with the same reference numerals. The invention relates to devices used to drain fluids from the body and more specifically relates to effusion drains.

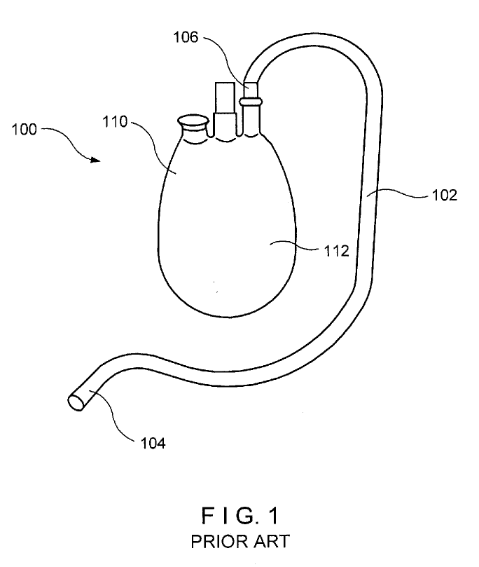

FIG. 1 shows an exemplary drainage device 100 comprising a drain tube 102 and a drainage bulb 110. The drain tube 102 has a distal end 104 that is inserted into the body to a target region so that fluid may enter the lumen of the drain tube 102 and flow therethrough to the drainage bulb 110. The drainage bulb 110 comprises a fluid connector 106 to couple with the drain tube 102, and a flexible balloon-like body 112.

The drainage bulb 110 provides a place to collect the fluid removed from the patient so that it can be measured and subsequently analyzed. In addition, the drainage bulb 110 provides the initial suction used to drain an initial fluid buildup in the target tissue. The user squeezes the drainage bulb 110 after the distal tip 104 has been inserted into the body and then releases it to create a negative pressure that draws fluid from the target region into the bulb 110.

The size of conventional drainage bulbs such as the bulb 110 make them difficult to store and inconvenient to wear. In particular, when the body 112 of such a drainage bulb fills with fluid, it may become heavy and difficult to manage. Furthermore, weak patients or those suffering from arthritis may find it difficult to compress the bulb 110 to create the initial vacuum and to connect the bulb 110 to the drain tube 102.

According to the exemplary embodiments of the present invention, a flat expandable effusion drain is provided that is easy to operate and which has a low profile to minimize inconvenience to the user. The compact effusion drain according to the invention comprises a passive valve system that seals the device when not in use and which self-limits the volume of the device limiting the amount of fluid collected at any time to prevent an excessive increase in the size and weight of a storage portion of the system. A one way valve is provided to prevent the fluid from flowing back into the patient.

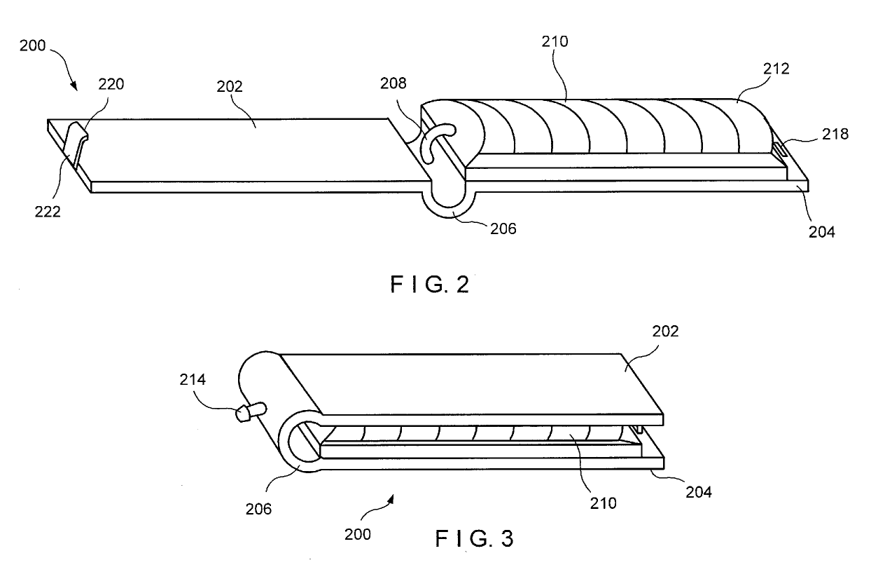

As shown in FIGS. 2 and 3, an effusion drain 200 according to an exemplary embodiment of the invention comprises a two portion outer clam shell formed of first and second shells 202, 204, respectively, connected by a living hinge 206. The first and second shells 202, 204 may be moved manually between the open and closed positions. In the open position (FIG. 2) the first and second shells 202, 204 lie flat substantially on the same plane, similar to an open book. To close the device, the first and second shells 202, 204 are rotated about the hinge 206 through approximately 180° such that opposite faces of the first and second shells 202, 204 are nearly parallel and face each other as shown in FIG. 3.

A fluid collection element such as an expandable pouch 210 is attached to one of the shells, in this case shell 204, to form a receptacle for drained fluids. The expandable pouch 210 is compressed to a minimum volume when the first and second shells 202, 204 of the effusion drain 200 are closed and is free to expand when the first shell 202 is rotated/pushed away from the second shell 204. A shape memory mechanism is included in the expandable pouch 210 to expand it to a maximum volume when not constrained between the first and second shells 202, 204. For example, the shape memory mechanism may comprise a set of resilient ribs 212 incorporated in the pouch 210 to provide the ‘memory’ required to expand the pouch 210 after it has been compressed enabling the expandable pouch 210 to create negative pressure drawing fluid through the drain tube eliminating the difficulties associated with systems which require the patient to compress a bulb to generate this negative pressure.

The effusion drain 200 comprises a connecting tube 208 fluidly connecting the expandable pouch 210 and a barb fitting 214 conveniently located to be accessible whether the effusion drain 200 is closed or open, so that a drainage tube may be connected thereto in either case. The barb fitting 214 preferably comprises a valve to prevent flow of fluids from the pouch 210 back into the patient. For example, a Pressure Activated Safety Valve (PASV), such as those referenced in U.S. Patent Application Publication Nos. 2006/0184139, 2005/0171510, 2005/0171490, 2005/0171489, and 2005/0171489, the disclosures of all of which are hereby expressly incorporated by reference in their entireties, may be located within the barb fitting 214 to selectively seal the lumen of the barb fitting and prevent fluid back flow. In addition, or as an alternative to the PASV valve, a one way valve may be included in the barb fitting 214 to prevent back flow therethrough.

A latching mechanism may be provided to retain the first and second shells 202, 204 in the closed position when desired. For example, a limited travel latch may be used, so that after the first and second shells 202, 204 are latched together, they are allowed to move apart from one another only by a set distance. A latch 220 may be provided on one of the first and second shells 202, 204 with a latch lock 218 on the other so that, once the latch 220 has been inserted into the latch lock 218, the first and second shells 202, 204 are retained in the closed configuration shown in FIG. 3.

The length of a latch body 222 is preferably selected to define a desired maximum volume of the expandable pouch 210 with a longer latch body 222 increasing the maximum latched distance between the first and second shells 202, 204 and, consequently, increasing the maximum volume of the expandable pouch 210. Initially the first and second shells 202, 204 are fully compressing the expandable pouch 210, and are then released by a given amount allowing the expandable pouch 210 to expand to a desired maximum volume. This expansion of the pouch 210 generates the negative pressure necessary to draw fluid into the effusion drain 200.

During use of the exemplary expandable effusion drain 200, the clamshell housing formed by the first and second shells 202, 204 is closed while attaching the drain tube to the barb fitting 214, compressing the expandable pouch 210 and collapsing it. This movement is easy to perform even for weak patients or patients suffering of arthritis, since the hinge 206 and the length of the first and second shells 202, 204 provide leverage. The patient closes the shells 202, 204 sufficiently for the latch 220 to engage the latch lock 218.

When the patient releases the device after latching it, the shape memory mechanism of the expandable pouch 210 expands the latter to the extent allowed by the length of the latch body 222. As the pouch 210 expands, negative pressure is generated that creates suction drawing fluid through the drainage tube into the pouch 210. The length of the latch body 222 may be selected to limit the maximum volume of the expandable pouch 210 to ensure that the device does not increase beyond a desired maximum weight or from expanding beyond a desired maximum volume.

In one exemplary embodiment according to the invention, the first and second shells 202, 204 are preferably formed of a plastic material, and may be molded as a single piece. However, those skilled in the art will understand that these components may be formed of any suitable material. Those skilled in the art will understand that the expandable pouch 210 with the reinforcing ribs 212 may be sealed to one of the first and second shells 202, 204 using, for example, heat or sonic energy. In the example shown, the barb with the PASV and the connecting tube may be solvent, heat or sonically bonded to the clamshell 202, 204. Those of skill in the art will understand that other manufacturing processes may be used to form the expandable effusion drain 200 and any components thereof.

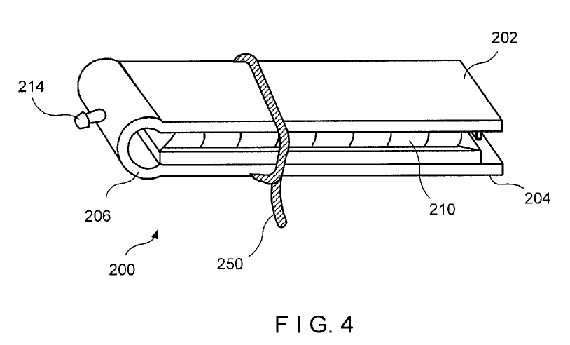

Another embodiment of the present invention comprises a retaining element selectively locking the first and second shells 202, 204 in the closed position. The retaining element may be used with or without the latch 220 and the latch lock 218. In the embodiment shown in FIG. 4, a ripcord 250 maintains the expandable pouch 210 compressed between the first and second shells 202, 204 while it remains in the package. Once the effusion drain 200 is unpacked and has been connected to a drainage catheter, the ripcord 250 is pulled to release the expandable pouch 210. The shape memory mechanism then expands the pouch 210 to its expanded form generating suction which draws fluid into the drainage catheter and, consequently, into the pouch 210. Thus, the user thus does not have to compress the device prior to use, further simplifying its operation. Those of skill in the art will understand that different releasable retaining elements may be used, such as clasps, friction locks, etc. Alternatively, packing material may maintain the drain 200 in the compressed configuration. Then, after the drain 200 has been connected to a drainage catheter, the packing material may be removed, allowing the pouch 210 to expand, generating suction to draw fluid into the drainage catheter and, consequently, into the pouch 210. As would be understood by those skilled in the art, the packaging or other structure maintaining the drain 200 in the compressed configuration may include a snap lock, a perforated sleeve, a twist lock or any other suitable releasable locking structure.

Furthermore, any of a variety of attachment devices may be used to secure an effusion drain according to any of the embodiments of the invention at a desired location. For example, a clip or belt may be used to attach the device to clothing with the attachment device connected, for example, to the first and second shells 202, 204 close to the living hinge 206.

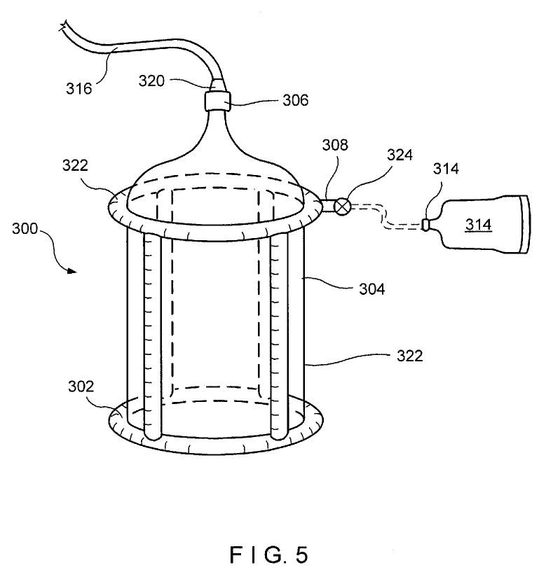

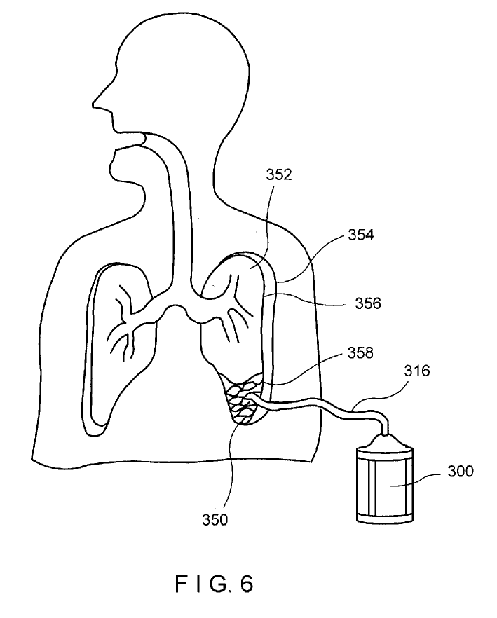

As shown in FIGS. 5 and 6, an inflatable vacuum drainage bag 300 according to a further embodiment of the invention is suitable, for example, for draining effusions such as pleural effusions accumulating between layers 354, 356 of the membrane that lines the lungs and the chest cavity. As shown in FIG. 6, as a pleural cavity 358 fills with fluid 350, the lung 352 is compressed potentially causing breathlessness, congestive heart failure, infection and pulmonary emboli. Many patients suffering from pleural effusions are fitted with a drainage tube or catheter to remove the fluid and are sent home. Pleural effusions often require daily or more frequent drainage, so that large numbers of drainage containers are used. Thus it is desirable facilitate storage by packaging the containers so that they occupy as little space as possible. Conventional drainage containers often are rigid bottles that are difficult and cumbersome to transport and store. The containers may be evacuated before use, so that when connected to a drainage tube the vacuum therein draws an initial amount of fluid from the anatomical cavity into the container.

According to the present invention, a flexible inflatable vacuum drainage bag is provided to remove fluids from an anatomical cavity through a drainage tube or catheter. The exemplary inflatable vacuum drainage bag according to the invention can be easily used by a patient at home, to self-drain an effusion with little or no assistance. The exemplary device can be packaged for storage and transport in a reduced volume deflated or collapsed state, so that it occupies very little room and does not have to be handled with particular care. The device can be manufactured to be a low cost, disposable item. For example, multiple inflatable vacuum drainage bags may be packaged with a drainage catheter kit, to provide a self-contained package, which the patient can bring home.

As shown in FIG. 5, the inflatable vacuum drainage bag 300 comprises a fluid collection element 304 made of a polymer film, for example PVC, polyurethane or other deformable material. The fluid collection element 304 is flexible when the device is in the collapsed state, and can be folded as necessary for packaging. A fluid connector 306 is provided to connect the interior of the fluid collection element 304 to an exterior fluid conduit, such as a drainage catheter 316. The fluid connector 306 may comprise a valve 320, which remains closed when not connected to the drainage catheter 316. As described above, the drainage catheter 316 may be used to drain fluids from a chest cavity or other anatomical cavity such as the peritoneal cavity (e.g., to relieve a pleural effusion or ascites).

An inflatable frame 302 is formed of a network of channels 322 integral with the fluid collection element 304. When inflated, the inflatable frame 302 expands the entire vacuum drainage bag 300 to an operative shape, opening the drainage bag 300 and creating a vacuum therein. For example, the fluid collection element 304 in the operative shape may resemble an inflated balloon, an expanded bag, a bottle or other closed container adapted to hold a liquid. The inflatable frame 302, when inflated, may define the outline of a cylinder, a sphere, a balloon or other three-dimensional container. It also has sufficient stiffness to substantially maintain the operative shape of the vacuum drainage bag 300 during use.

The inflatable frame 302 is typically inflated after forming a fluid connection with the drainage catheter 316, so that the vacuum formed in the fluid collection element 304 motivates an initial fluid drainage from the anatomical cavity through the drainage catheter 316. Additional fluid may then drain naturally into the fluid collection element 304. In one exemplary embodiment, the expanded vacuum drainage bag 300 may have a volume of about 1 to 2 liter.

The inflatable frame 302 may comprise an optional inflation connector 308 to which an inflation source 310 can be connected. A corresponding connector 314 of the inflation source 310 may comprise a needle, a nozzle or any other convenient mechanism to form a coupling with the inflation connector 308. As would be understood by those skilled in the art, the inflation source may comprise a foot operated pump, a hand operated syringe or other device capable of providing pressurized air such as a small cylinder of compressed gas. Those of skill in the art will understand that other fluids in addition to air may be used to fill the inflatable frame 302. If multiple strokes of the inflation source 310 are necessary, such as when a syringe is used, a valve 324 may be provided to permit refilling the syringe while preventing the fluid from escaping the inflatable frame 302.

When the inflatable vacuum drainage bag 300 has been filled, or when sufficient fluid has been drained from the anatomical cavity to relieve the symptoms, the device may be detached from the drainage catheter 316 and disposed of. For example, the valve 320 may be used to seal the device from the outside environment.

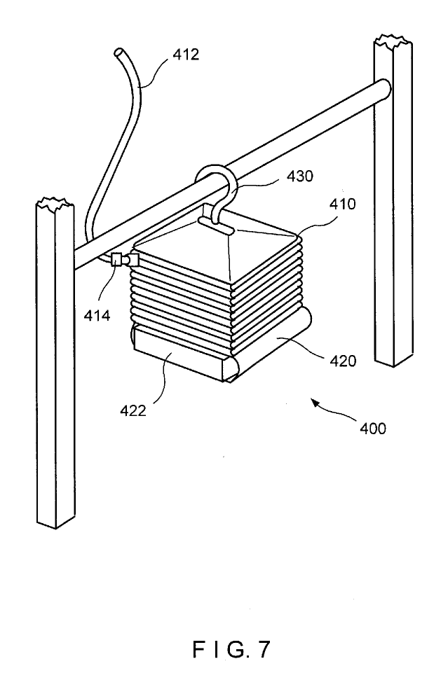

As shown in FIGS. 7-9, a collapsible, disposable drainage bag 400, according to a further embodiment of the present invention, is also suitable for draining effusions. The disposable drainage bag 400 may be connected to a drainage tube or drainage catheter 412 that is already indwelling in a patient as would be understood by those skilled in the art. The drainage bag 400 is collapsible into a very manageable size so that multiple bags may be stored in a single box and sent home with the patient. Another advantage is that the patient can easily drain the bag after use and discard the disposable bag 400.

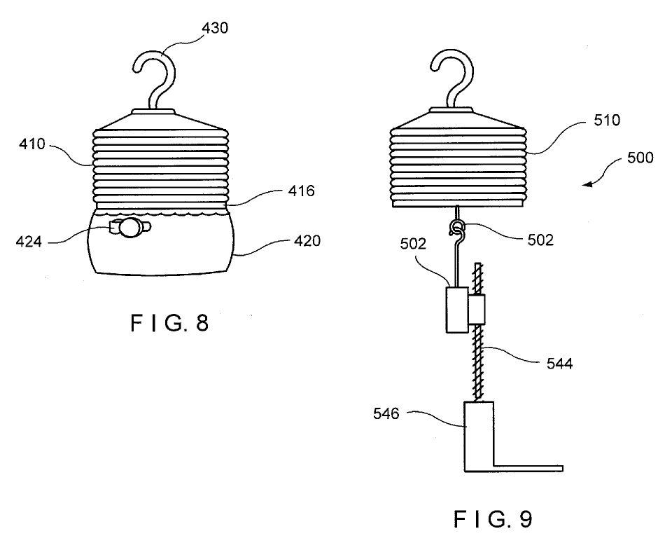

As shown in FIGS. 7 and 8, the disposable drainage bag 400 is comprised of two primary sections, an upper section 410 and a lower section 420, made of a polymer film, for example, polyurethane, polyethylene, polypropylene, or other standard material. The upper section 410 is the fluid drainage cavity that only communicates with the drainage tube 412 through its connector 414 to create a drainage cavity. The top of the drainage bag 400 also preferably includes a hanger 430 or other connector that may be used to hang the bag 400 from a side of a chair, bed, custom rack, or other similarly convenient item. The entire drainage bag 400, including the hanger 430, lies flat when in its collapsed state.

In one embodiment, as shown in FIG. 7, the lower section 420 acts as a pocket or a sleeve designed to receive a preexisting object with a known weight 422. After the collapsed drainage bag 400 is removed from its package and connected to the drainage catheter 412 via the connector 414, the bag is hung next to the patient from the hanger or hook 430 and the weight 422 is inserted into the pocket of the lower section 420. The weight 422 pulls down on the lower section 420 of the drainage bag 400, expanding the upper section 410 and creating a vacuum inside the drainage cavity. The vacuum condition causes bodily fluid to flow from the patient through the drainage tube 412 and into the upper section 410. Once the drainage bag 400 is full, or drainage is complete for that setting, the drainage bag 400 and the drainage catheter or drainage tube 412 are closed off from the atmosphere and disconnected. The weight 422 is removed and saved for reuse with a new disposable drainage bag 400.

In an alternative embodiment, shown in FIG. 8, the lower section 420 is a sealable cavity with an inlet port 424. The inlet port 424 may be used to fill the lower section 420 with water or other liquids to provide the necessary weight to expand drainage cavity of the upper section 410 thus avoiding the need to carry a weight along with the drainage bags. A rigid plate 416 may also be included in the septum, between the upper section 410 and the lower section 420, to facilitate even filling and expansion of the upper section 410.

As shown in FIG. 9, a drainage bag 500 according to a further embodiment of the invention comprises a single section 510, which functions in substantially the same way as the upper section 410, described in previous embodiments. However, in addition to this functionality, a rigid floor 516 of the single section 510 includes a loop or hook 518 attached to a bottom outside surface thereof. Once the drainage bag 500 is connected to a drainage tube or catheter, the section 510 is mated with a mechanical rod comprising a motor 542 and a coupling (e.g., a threaded shaft 544) and a stand 546. The motor 542 and the threaded shaft 544 are mounted on the stand 546, which has an adjustable height. The motor 542 is mated with the loop 502 so that as the motor 542 drives itself down the threaded shaft 544, the drainage cavity of section 510 opens at a controlled constant rate. The vacuum created in section 510 facilitates fluid flow from the patient to the drainage bag 500 at a desired rate via a drainage tube.

In an alternative embodiment, an elastic member compressible to a flat state and which has a normally open condition is incorporated into the fold of the single section 510. Once the drainage bag 500 is connected to the drainage tube or connector in its collapsed form (e.g., under compression), the compression is released to create a vacuum to drain fluid from the patient, making a mechanical rod unnecessary for this embodiment. In another embodiment, the compressible material incorporated into the fold of the single section 510 includes a member formed of a shape memory material (e.g., Nitinol) which, when activated (e.g., electrically or via temperature change), reverts to a memorized shape which decompresses the single section 510 to create the vacuum and drain fluids as desired. For example, as would be understood by those skilled in the art, a shape memory material may be selected which is activated through warming under the influence of bodily fluids at approximately 36° C.

In another embodiment, an electro-activate polymer (EAP) member may be incorporated into the fold of single section 510 such that the drainage bag 500 so that, when the EAP member is not activated, the single section 510 is compressed flat. As would be understood by those skilled in the art, the EAP member is formed so that, when an electric charge is administered, the EAP member reverts to a memorized shape which expands the single section 510 create a vacuum facilitating fluid flow thereinto.

The present invention has been described with reference to specific embodiments. However, other embodiments may be devised that are applicable to other medical devices, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

As described above, the present invention is directed to an effusion drain comprising first and second shell members hinged together and pivotable relative to one another between an open and a closed position; a fluid collection element disposed between the first and second shell members; and a shape memory device biasing the fluid collection element to an expanded volume configuration.

In another embodiment, the effusion drain further comprises a locking mechanism maintaining the first and second shell members in the closed configuration. In a more specific embodiment of the present invention, the locking mechanism limits travel of the first and second shell members relative to one another beyond a predetermined extent and may include a latch.

The method according to a specific embodiment of the invention comprises inserting one end of a catheter to a target anatomical region within a body; closing a clamshell body of an effusion drain coupled to the catheter to compress an expandable fluid collection element included within the clamshell body; and releasing the latch to allow a bias of a shape memory element to expand the fluid collection element to generate a suction through the catheter to draw fluids into the fluid collection element.

In another embodiment, the present invention is directed to an effusion drain comprising a fluid collection element moving between an operative shape for receiving a fluid and a reduced volume collection element for connecting to a fluid drain; a lower section coupled to the fluid collection element and adapted for receiving a weight, such that the fluid collection element is expanded to the operative shape; and a hook attached to the top of the fluid collection element.

In a more specific embodiment, the lower section comprises a pocket adapted to receive an object of a known weight.

According to another embodiment the method comprises inserting a distal end of a drainage catheter into an anatomical cavity within a body; forming a connection between a vacuum drainage bag and a proximal end of the drainage catheter; and weighing down a lower section coupled to the vacuum drainage bag to expand the drainage bag to an operative shape, expansion of drainage bag forming a vacuum therein to draw an initial portion of fluid thereinto.

What is claimed is:

1. An effusion drain, comprising:
a fluid collection element moving between an operative shape for receiving a fluid and a reduced volume shape for connecting to a fluid drain;
a compressible member biasing the fluid collection element to the operative shape, the compressible member being incorporated into the fluid collection element such that release of a compression on the compressible member expands the fluid collection element to the operative shape.

2. The effusion drain of claim 1, further comprising first and second shell members hinged together and pivotable relative to one another between an open and a closed position, the fluid collection element disposed between the first and the second shell members.

3. The effusion drain of claim 2, further comprising a locking mechanism maintaining the first and the second shell members in a closed configuration and limiting travel of the first and the second shell members relative to one another beyond a predetermined extent.

4. An effusion drain, comprising:
a fluid collection element movable between an operative shape for receiving a fluid and a reduced volume shape for connecting draining fluid therefrom;
an inflatable frame coupled to the fluid collection element so that, as the inflatable frame moves between a collapsed shape and an inflated shape, the fluid collection element is expanded to the operative shape, the inflatable frame including an inflation connector for connecting a source to a source of inflation fluid.

5. An effusion drain, comprising:
a fluid collection element movable between an operative shape for receiving a fluid and a reduced volume shape for draining fluid therefrom;
a lower section for supplying force to the fluid collection element expanding the fluid collection element into the operative shape; and
a hook attached to a top portion of the fluid collection element.

6. The effusion drain of claim 5, the lower section comprising a sealable cavity with an inlet adapted to receive a source of inflation fluid.

7. The effusion drain of claim 5, the lower section comprising a mechanical rod matable to the fluid collection element such that a motor, connected to a threaded shaft mounted on a stand, drives itself down the threaded shaft and expands the fluid collection element to its operative shape.

8. A method of draining biological fluids, comprising:
inserting a distal end of a drainage catheter to a target anatomical region within a body;
forming a connection between a fluid collection element and a proximal end of the drainage catheter; and
releasing a compressible member incorporated into the fluid collection element such that the fluid collection element expands to an operative shape, suction generated in the catheter thereby drawing fluids into the fluid collection element.

9. The method of claim 8, wherein releasing the compressible member includes using one of a temperature stimulus and an electric charge to trigger the release of the compressible member.

10. A method of draining biological fluids, comprising:
inserting a distal end of a drainage catheter into an anatomical cavity;
forming a connection between a fluid collection element and a proximal end of the drainage catheter; and
inflating an inflatable frame coupled to the fluid collection element to expand the fluid collection element to an operative shape forming a vacuum therein to draw an initial portion of fluid thereinto.

11. A method for draining a fluid, comprising:
inserting a distal end of a drainage catheter into an anatomical cavity within a body;
forming a connection between a fluid collection element and a proximal end of the drainage catheter; and
supplying force to a lower section coupled to the fluid collection element to expand the fluid collection element to an operative shape forming a vacuum therein to draw an initial portion of fluid thereinto.

12. The method of claim 11, further comprising inflating a sealable cavity of the lower section with an inflating fluid to weigh down the lower section.

13. The method of claim 11, wherein supplying the force to the lower section includes actuating a mechanical rod of the lower section such that a motor drives itself down a threaded shaft which is mounted on a stand, expanding the fluid collection element to an operative shape and forming a vacuum therein to draw an initial portion of fluid thereinto.

14. The method of claim 13, wherein the mechanical rod of the lower section is coupled to the fluid collection element via a ring attached to a rigid floor of the fluid collection element.
